(19) United States
(12) Patent Application Publication
(10) Pub. No.: US 2012/0041283 A1
(43) Pub. Date: Feb. 16, 2012

(54) DEVICE FOR EVALUATING CONDITION OF SKIN OR HAIR

(75) Inventors: Srinivasan Krishnan, Stamford, CT (US); Jamie Gordon Nichol, Arlington, MA (US); Bruce Cichowlas, Framingham, MA (US)

(73) Assignee: CONOPCO, INC., D/B/A UNILEVER, Englewood Cliffs, NJ (US)

(21) Appl. No.: 12/855,728

(22) Filed: Aug. 13, 2010

(51) Int. Cl. A61B 6/00

(52) U.S. Cl. 600/306

(57) ABSTRACT

A hand-held device for evaluating skin or hair condition which includes a housing, a hydration meter, and a plurality of light emitting diodes (LEDs). The hydration meter is supported within the housing and has an external surface portion contactable against skin or hair to measure moisture content. At least one of the LEDs both emits and absorbs light. Advantageously the LEDs include a red, a blue, a green and two infrared wavelength light emitters.

### BACKGROUND OF THE INVENTION

1. Field of the Invention

The invention concerns a hand holdable device for simultaneously measuring multiple parameters defining a person's skin or hair condition.

2. The Related Art

Most people do not have perfect skin or hair. Imperfections can cosmetically be treated with an appropriate product. Selection of the appropriate product is often not scientific. Some will provide excellent results, others modest improvement, and still others will simply not be effective. A correct match needs to start with an evaluation of a person's skin or hair condition.

Evaluation systems have been reported in the literature. For instance, U.S. Pat. No. 5,622,692 (Rigg et al.) discloses a hand-held device for measuring skin color at a point of retail sale and recommending a suitable facial foundation. U.S. Pat. No. 5,945,112 (Flynn et al.) discloses a method for providing a customized skin foundation product to cover human skin imperfections. The steps include spectrophotometrically measuring a customer's normal skin to obtain normal skin coloration values of lightness, redness and yellowness. These coloration values are then converted through calculation to a modified value determined by a set program. The skin analyzing module is a hand-held spectrophotometer/colorimeter operating with at least one visible light source such as an LED operating in the 400-900 nm wavelength range.

JP 2003210416 (Wave Cyber KK) describes a skin measuring instrument held within a transparent body. A light source shines illuminating light through the transparent body and an optical detector absorbs reflected light from the contact surface. Water content is measured by way of static capacitance at a skin contact point. Also present within the body is a camera for photographing the skin surface.

Although devices to measure properties of skin or hair are known, none deliver a comprehensive evaluation. Better devices are required which simultaneously measure/deliver information on moisture content, blemishes and topography.

### SUMMARY OF THE INVENTION

A device for evaluating skin or hair condition is provided which includes:
(i) a housing;
(ii) a hydration meter for measuring moisture, the meter being supported within the housing and having an external surface contactable against skin or hair to measure moisture content thereof, the meter including at least two adjacent metallic wires with their respective capacitance sensitive to differences in dielectric constant; and
(iii) a plurality of light emitting diodes arranged within the housing wherein at least one of the light emitting diodes at different times emitting light and also absorbing light reflected from the skin or hair area being evaluated.

### BRIEF DESCRIPTION OF THE DRAWING

Further advantages and features of the present invention will become more apparent from consideration of the drawing wherein:

FIG. 1 is a perspective view of the first embodiment;

FIG. 2 is a top plan view of the first embodiment; and

FIG. 3 is a diagrammatic representation of the LED arrangement.

### DETAILED DESCRIPTION OF THE INVENTION

Now we have provided a device which can more fully evaluate skin or hair condition. This device combines a hydration meter measuring moisture with a light based system measuring color and topography. The device is highly portable. Moreover, the collected data can via a cord terminated by a Universal Serial Bus (USB) connector be readily downloaded to a computer. A skin or hair value is then calculated based upon the downloaded data.

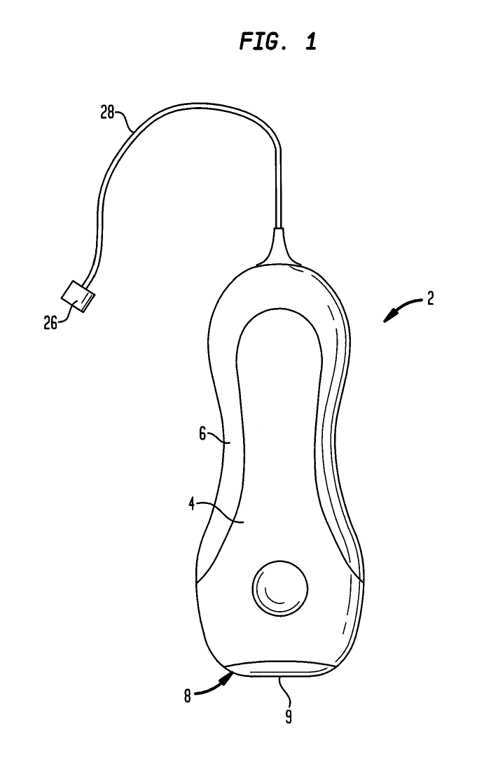

FIG. 1 illustrates one embodiment of the hand-held device. By the term “hand-held” is meant a device measuring in length less than 35 cm, preferably between 10 and 25 cm (not including cord) and a width between 2 and 8 inches, preferably between 3 and 6 cm. The device features a housing 2 with a shell 4 and a gripping portion 6. Normally the shell is formed of a relatively hard plastic such as ABS (polyacrylonitrile-butadiene-styrene) which is a high impact resistant plastic. Advantageously the gripping area is formed of a less rigid material such as a rubber.

Measurements are taken from an evaluation area 8 of the device. Normally area 8 is on an end of the device distant from any electrical or fiber optic cords exiting the housing. Area 8 has at least a central section formed of a transparent wall. Clear plastic is useful for this purpose. Light transmission through the clear plastic is a necessary requirement.

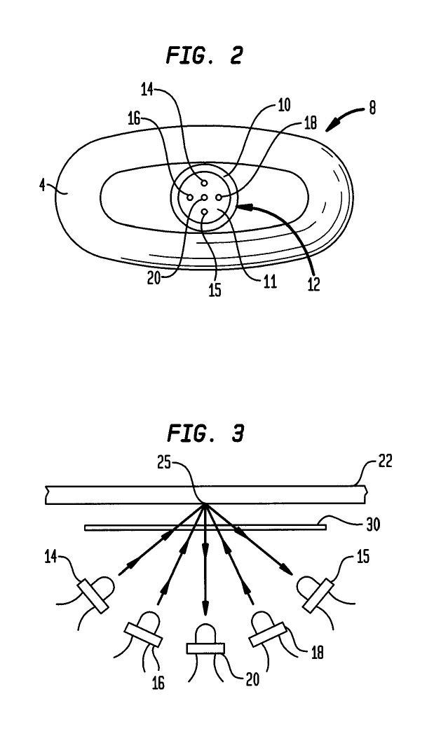

FIG. 2 best illustrates the evaluation area 8. When this evaluation area is contacted against skin, the first contact surface is an external surface 9 of a moisture sensing cell 10 of a hydration meter 12. The sensing cell 10 picks up electrical signals from the stratum corneum of skin. Metallic electrical conducting wires, preferably of copper on a circuit board, are embedded within a hardened resin of the sensing cell. These wires are sensitive to differences in dielectric constant of their aqueous environment. Differences in relative electrical capacitance resulting from differences in the dielectric constant reveal the measure of moisture at the skin or hair surface. Hydration meters are commercially available from Courage-Khazaka Electronics, Koln, Germany. The sensing cell 10 is circular (donut shaped) having a central transparent window 11 through which light can be transmitted.

A plurality of light emitting diodes (LEDs) are positioned as a cluster of five within the housing interior to the hydration meter sensing cell 10. These provide input on amount and distribution of melanin. FIG. 2 reveals a red wavelength LED 14 paired with an infrared LED 15; a blue wavelength LED 16; a green wavelength LED 18; and a central absorbing infrared wavelength LED 20. Respective wavelength ranges are for the red (650-560, preferably about 570 nm), green (530-555, preferably about 540 nm), blue (415-440, preferably about 430 nm) and infrared (850-900, preferably about 880 nm). Advantageously the blue, green and red LEDs have their tips angled to transmit light at an angle between 20° and 75°, preferably 35°-55°, and optimally about 45° relative to the central window and to the skin. In other words, these LEDs transmit light at preferentially about a 45° angle to the skin. In a preferred embodiment, the central infrared LED 20 is focused at approximately a 90° orientation (perpendicular) to the central window and to the skin surface.

An important aspect of the present invention is that one or more of the LEDs perform a dual function of both emitting and absorbing reflected light. These LEDs essentially are two-way conduits of light. Their light impinges a skin area 22 and also receives reflection from that area. FIG. 3 best illustrates the LED arrangement.

In the preferred embodiment, reflectance spectroscopy is utilized to evaluate color and texture of the analyzed skin. This means light is bounced off the skin and strength of the reflection is measured. LEDs are utilized because they are photo diodes that can evaluate shorter wavelengths. In the preferred embodiment, one or more of the LEDs emit and detect, but not simultaneously. Some of the LEDs in the device only emit light, others only detect and in the preferred embodiment the central infrared LED performs both functions.

Reflectance spectroscopy measurements can be confused by gloss on the skin surface. The special arrangement of the LEDs in the sensor allows measurement of surface gloss as well as the spectro signature of the skin.

The four LEDs that surround the central infrared LED 20 are arranged so that the axis of each intersects at point 25 where the axis of the central infrared LED impinges at a surface of the skin.

Measurements are taken as follows. The red LED 14 is placed opposite the infrared LED 15. Two measurements of light intensity are taken using the central infrared LED 20 and the infrared LED 15. These two measurements provide input on the spectro/diffuse surface characteristics, and red light amounts the surface absorbs.

Thereafter, the red LED 14 is turned off. Now the green LED 18 is illuminated. A single measurement is taken using the central infrared LED 20. The same sequence is repeated for the blue LED 16 and the infrared LED 15. Almost simultaneously the hydration meter 12 via the moisture sensing cell 10 measures capacitance around or concentric with the illuminated skin surface.

Data generated from the hydration meter and the LEDs initially is in analog form. This form arises because the meter and LEDs are transducers which inherently provide an analog response to the physical event being measured. The analog data is next converted to digital values (numbers stored in a micro-controller). Thereafter, the digital values are transformed back into analog mode, this time as an audio wave signal. The audio signal is thereafter transformed back into a digital signal at a downstream USB microchip. By this manner we can double the base frequency or reduce the base frequency by half. In summary, the data is converted from analog to digital to analog to digital. This ADAD conversion is a key factor allowing use of off-the-shelf components to transfer skin data in a way the computer already recognizes.

The generated data is converted through a pre-set series of calculations to identify a Skin Index unique to the measured skin area. The Skin Index permits a user to monitor their skin over a period of time. A product recommended by the program to adjust the consumer's skin into an improved condition may be applied over the monitored period. This allows a consumer to evaluate effectiveness of the product or any other products that might be applied to improve the skin condition.

A USB port 26 is attached to an end of an electrical wire or optical fiber cord 28 as shown in FIG. 1. The USB port can plug into a computer to access a program companion to the device. It is the most advantageous connector to the computer. However, the system may also work by Bluetooth connectivity or serial port connection routes.

Most preferred is that power to the system be delivered externally from an electric grid. Alternatively, power can be supplied by a rechargeable battery or disposable batteries within the device. In circumstances wherein power is supplied by a rechargeable or a disposable battery, the only cord projecting from the housing is the data cord 28.

In one aspect of the invention, the device can be considered as a “personal trainer” for a user's skin.

A polarizer plate 30 may be placed adjacent the central window through which all the LEDs transmit/absorb light.

All documents referred to herein, including all patents, patent applications, and printed publications, are hereby incorporated by reference in their entirety in this disclosure.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

It should be noted that in specifying any range of concentration or amount, any particular upper concentration can be associated with any particular lower concentration or amount.

The foregoing description illustrates selected embodiments of the present invention and in light thereof variations and modifications will be suggested to one skilled in the art all of which are within the spirit and purview of this invention.

What is claimed is:

1. A device for evaluating skin or hair condition comprising:
(i) a housing;
(ii) a hydration meter mechanism for measuring moisture, the meter being supported within the housing and having an external surface contactable against skin or hair to measure moisture content thereof, the meter comprising at least two adjacent metallic wires with their respective capacitance sensitive to differences in dielectric constant; and
(iii) a plurality of light emitting diodes arranged within the housing wherein at least one of the light emitting diodes at different times emitting light and absorbing light reflected from the skin or hair area being evaluated.

2. The device according to claim 1 wherein the light emitting diodes comprise one red, one blue, one green and two infrared wavelength light emitters.

3. The device according to claim 1 wherein the external surface portion of the hydration meter surrounds a central window, the light emitting diodes being arranged to transmit light through the central window.

4. The device according to claim 3 wherein the light emitting diodes are angularly oriented to the central window in an angular range from 20° to 90°.

5. The device according to claim 4 wherein the angular range is from 35° to 55°.

6. The device according to claim 3 wherein one of the plurality of light emitting diodes is an infrared wavelength receiver oriented perpendicular to the central window and the skin or hair being measured.

7. The device according to claim 1 comprising a single cord bearing an optical fiber or electrical wire for transmission of data and that exits the housing, and at an end of the cord having a USB port.

8. The device according to claim 7 wherein the hydration meter mechanism and light emitting diodes generate data in analog form, and wherein the data in analog form is converted to digital values and then retransformed into an analog form as an audio signal for transmission.
